(19) United States
(12) Patent Application Publication
(10) Pub. No.: US 2014/0024913 A1
(43) Pub. Date: Jan. 23, 2014

(54) NEUROPHYSIOLOGIC PERFORMANCE MEASUREMENT AND TRAINING SYSTEM

(71) Applicants: Bryan D. Hixson, Thousand Oaks, CA (US); Dale Dalke, Thousand Oaks, CA (US)

(72) Inventors: Bryan D. Hixson, Thousand Oaks, CA (US); Dale Dalke, Thousand Oaks, CA (US)

(73) Assignee: NEUROTOPIA, INC., Thousand Oaks, CA (US)

(21) Appl. No.: 13/552,347

(22) Filed: Jul. 18, 2012

(51) Int. Cl. A61B 5/0478

(52) U.S. Cl. 600/383

(57) ABSTRACT

Preferably, an embodiment of an apparatus includes at least a plurality of sensor assemblies, wherein each sensor assembly provides at least one electrically responsive surface, and an oscillation device communicating with the sensor assembly. Preferably, the sensor assembly includes at least a signal processing circuit in electrical communication with the oscillation device to selectively agitate the at least one electrically responsive surface. The preferred apparatus further included a brainwave processing system communicating with each of the plurality of sensor assemblies, and a ground reference interacting with the brainwave processing system, wherein a selected one of the plurality of sensor assemblies provides a reference signal for each of the remaining sensor assemblies, and in which each electrically responsive surface is in pressing contact with a cranium of a subject.

### FIELD OF THE INVENTION

The present invention relates to the field of sensors. More particularly, the present invention relates to measurement and training systems for use in collecting brainwave data from subjects, and altering the brain state of the subject to obtain pick mental performance prior to the subject engaging in an activity.

### BACKGROUND OF THE INVENTION

Prior art sensor probe assemblies, have for the most part, depended on the preparation of an area of interest on a cranium of a subject, application of a gel like conductive material, and attachment of the probe to the cranium of the subject at the prepared and gelled site.

As advancements have been made in the field of electronics, it has become desirable to obtain neurophysiological signal data from subjects external to a laboratory or testing facility environment, without the need to prepare and apply a gel to a site of interest. Accordingly, improvements in apparatus and methods of providing dry sensors are needed, and it is to these needs the present invention are directed.

### SUMMARY OF THE INVENTION

In accordance with preferred embodiments, preferably, an embodiment of an apparatus includes at least at least a plurality of sensor assemblies, wherein each sensor assemblies providing at least one electrically responsive surface, and an oscillation device communicating with the sensor assembly. Preferably, the sensor assembly includes at least a signal processing circuit in electrical communication with the oscillation device to selectively agitate the at least one electrically responsive surface. The preferred apparatus further included a brainwave processing system communicating with each of the plurality of sensor assemblies, and a ground reference interacting with the brainwave processing system, wherein a selected one of the plurality of sensor assemblies provides a reference signal for each of the remaining sensor assemblies, and in which each electrically responsive surface is in pressing contact with a cranium of a subject.

An alternate preferred embodiment, includes at least the steps of providing a plurality of sensor assemblies, in which each sensor assembly includes at least one electrically responsive surface, and supplying an oscillation device for communication with each sensor assembly. Preferably, each sensor assembly includes at least a signal processing circuit in electrical communication with the oscillation device, and the oscillating device selectively agitates at least one electrically responsive surface. The preferred method further includes steps of communicating performance measurement data from at least one of the plurality of sensor assemblies to a brainwave processing system, and furnishing a ground reference, which preferably interacts with the brainwave processing system. In the preferred method, one of the plurality of sensor assemblies is selected to provide a reference signal for each of the remaining sensor assemblies, and in which each electrically responsive surface is in pressing contact with a cranium of a subject.

These and various other features and advantages that characterize the claimed invention will be apparent upon reading the following detailed description and upon review of the associated drawings.

### BRIEF DESCRIPTION OF THE DRAWINGS

The present invention is illustrated, by way of example and not limitation, in the accompanying drawings, like references indicate similar elements in which:

FIG. 1 is a top plan view of an embodiment exemplary of the inventive sensor probe assembly.

FIG. 2 is a view in elevation of an embodiment exemplary a conductive pin of the inventive sensor probe assembly of FIG. 1.

FIG. 3 is a front side view in elevation of an embodiment exemplary of the inventive sensor probe assembly of FIG. 1.

FIG. 4 is a front side view in elevation of an embodiment exemplary of the inventive sensor probe assembly illustrative of a flexible, electrically conductive pin securement member and associated plurality of electrically conductive pins matted thereto, of an embodiment exemplary of the inventive sensor probe assembly of FIG. 1.

FIG. 5 is a top plan view of an alternate embodiment exemplary of the inventive sensor probe assembly.

FIG. 6 is a view in front elevation of an alternate embodiment exemplary an electrically conductive pin of the inventive sensor probe assembly of FIG. 5.

FIG. 7 is a front side view in elevation of an alternate embodiment exemplary of the inventive sensor probe assembly of FIG. 5.

FIG. 8 is a front side view in elevation of an alternate embodiment exemplary of the inventive sensor probe assembly illustrative of a flexible, electrically conductive pin securement member and associated plurality of electrically conductive pins matted thereto, of an embodiment exemplary of the inventive sensor probe assembly of FIG. 5.

FIG. 9 is a front elevation view of an embodiment exemplary of an electrically conductive pin of FIG. 6, showing a head portion, a tip portion, and a body portion disposed there between.

FIG. 10 is a front elevation view of an embodiment exemplary of an electrically conductive pin of FIG. 2, showing a head portion having a convex shape, a tip portion, and a body portion disposed there between.

FIG. 11 is a front elevation view of an alternate embodiment exemplary of an electrically conductive pin of FIG. 2, showing a head portion having a concave shape, a tip portion, and a body portion disposed there between.

FIG. 12 is a front elevation view of an embodiment exemplary of an electrically conductive pin of FIG. 2, showing a head portion having a substantially flat top surface, a tip portion, and a body portion disposed there between.

FIG. 13 is a partial cutaway front elevation view of an alternate tip configuration for any of the electrically conductive pins of FIG. 9, 10, 11, or 12.

FIG. 14 is a cross-section, partial cutaway front elevation view of an alternate tip configuration for any of the electrically conductive pins of FIG. 9, 10, 11, or 12.

FIG. 15 is a partial cutaway front elevation view of an alternative tip configuration for any of the electrically conductive pins of FIG. 9, 10, 11, or 12.

FIG. 16 is a partial cutaway front elevation view of an alternate tip configuration for any of the electrically conductive pins of FIG. 9, 10, 11, or 12.

FIG. 17 is a flowchart of a method of producing an embodiment exemplary of the inventive sensor probe assembly of either FIG. 1 or FIG. 5.

FIG. 18 is a front elevation view of an embodiment exemplary of the present novel sensor assembly.

FIG. 19 is a bottom plan view of the novel sensor assembly of FIG. 18.

FIG. 20 is a front elevation, exploded view of the novel sensor assembly of FIG. 18.

FIG. 21 is a front elevation view of an alternate embodiment exemplary of the present novel sensor assembly.

FIG. 22 is a side elevation view of an alternate embodiment exemplary of the present novel sensor assembly of FIG. 21.

FIG. 23 is a side elevation view of an alternate embodiment exemplary of the present novel sensor assembly of FIG. 21, communicating with a brainwave processing system.

FIG. 24 is a schematic of a preferred signal processing circuit of the embodiment exemplary of the present novel sensor assembly of either FIG. 18, 21, or 23.

FIG. 25 is a flowchart of a method of using an embodiment exemplary of the inventive sensor assembly of either FIG. 18, 21, or 23.

FIG. 26 is a front elevation, exploded view of the alternative embodiment exemplary novel sensor assembly of FIG. 26, configured to support an oscillation device.

FIG. 27 is a front elevation view of an alternative embodiment exemplary of the present novel sensor assembly, configured to support an oscillation device.

FIG. 28 is a side elevation view of the alternative embodiment exemplary of the present novel sensor assembly of FIG. 26, configured to support an oscillation device.

FIG. 29 is a side elevation view of the alternative embodiment exemplary novel sensor assembly of FIG. 26, configured to support an oscillation device, and communicating with a brainwave processing system.

FIG. 30 is a front elevation, exploded view of an alternate alternative embodiment exemplary of the present novel sensor assembly, configured to support an oscillation device and a capacitance probe assembly.

FIG. 31 is a front elevation, cross-section view of the alternate alternative embodiment exemplary of the present novel sensor assembly of FIG. 30, configured to support an oscillation device and having the capacitance probe assembly attached thereto.

FIG. 32 is a side elevation, cross-section view of the alternate alternative embodiment exemplary of the present novel of FIG. 30, with the capacitance probe assembly secured thereon.

FIG. 33 is a bottom plan view of the alternate alternative embodiment exemplary of the present novel sensor assembly of FIG. 30.

FIG. 34 is a side elevation view of the alternate alternative embodiment exemplary of the present novel sensor assembly of FIG. 30, with the oscillation device and capacitance probe assembly attached thereto, and communicating with a brainwave processing system.

FIG. 35 is a schematic of the preferred alternate alternative embodiment exemplary of the capacitance probe assembly of the present novel sensor assembly of FIG. 34.

FIG. 36 is a flowchart of a method of using the alternate alternative embodiment exemplary of the present novel sensor assembly of FIG. 30, with the oscillation device and capacitance probe assembly attached thereto.

FIG. 37 is a schematic of the preferred alternative embodiment exemplary of the conductive probe assembly of the present novel sensor assembly of FIG. 29.

FIG. 38 is a schematic of the preferred alternate alternative embodiment exemplary of the capacitance probe assembly of the present novel sensor assembly of FIG. 34.

FIG. 39 shows a preferred configuration of an inventive standalone neurophysiologic performance measurement and training system, which preferably includes at least four sensor assemblies supported by a sensor assembly web.

### DESCRIPTION OF PREFERRED EMBODIMENTS

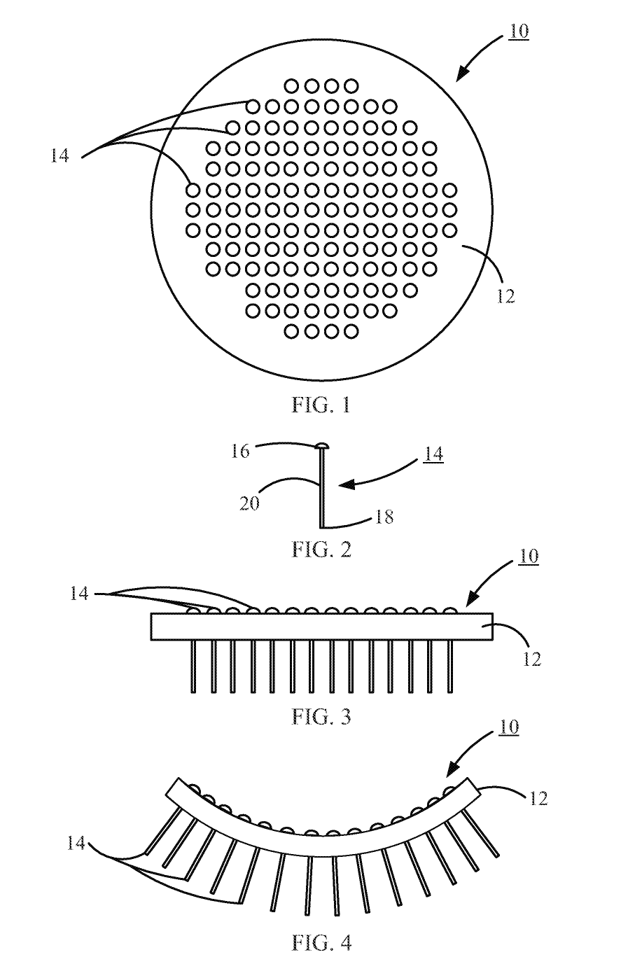

It will be readily understood that elements of the present invention, as generally described and illustrated in the figures herein, could be arranged and designed in a wide variety of different configurations. Referring now in detail to the drawings of the preferred embodiments, a sensor probe assembly 10, of FIG. 1, (also referred to herein as assembly 10) of a first preferred embodiment, while useable for a wide variety of bio-physiological sensing applications, it is particularly adapted for use as neurophysiological signal sensor component. Accordingly, the assembly 10 of the first preferred embodiment, of FIG. 1, will be described in conjunction with the merits of the use of the sensor probe assembly 10 as a neurophysiological signal sensor component.

In a preferred embodiment of FIG. 1, the sensor probe assembly 10 includes at least a conductive pin securement member 12, which hosts a plurality of conductive pins 14. Preferably, the plurality of conductive pins 14 are electrically conductive, and when in pressing contact with the conductive pin securement member 12, as shown by FIG. 3, form the sensor probe assembly 10 that yields a low impedance neurophysiological signal sensor component.

In a preferred embodiment, the conductive pins 14, an example of which is shown by FIG. 2, include at least a head portion 16, a tip portion 18, and a body portion 20 disposed between the head portion 16 and the tip portion 18. Preferably, each conductive pin 14 is formed from a non-corrosive material, such as stainless steel, titanium, bronze, or a gold plating on a rigid substrate selected from a group including at least polymers and metals. Preferably, the head portion 16 has a diameter greater than the diameter of the body portion 20.

As shown by FIG. 4, the conductive pin securement member 12 is preferably flexible and formed from a polymer. The electrical conductivity of the conductive pin securement member 12 is preferably attained by the inclusion of conductive particles embedded within the polymer. One such combination is a carbon filed silicon sheet material provided by Stockwell Elastomerics. Inc. of Philadelphia, Pa. However, as known in the art, conductive polymers may be formed from a plurality of polymer materials filled with conductive particles, the shape of which may be formed using well known manufacturing techniques that include at least molding, extrusion dies and sliced to thickness, formed in sheets and: die cut; cut with hot wire equipment; high pressure water jets, or steel rule dies.

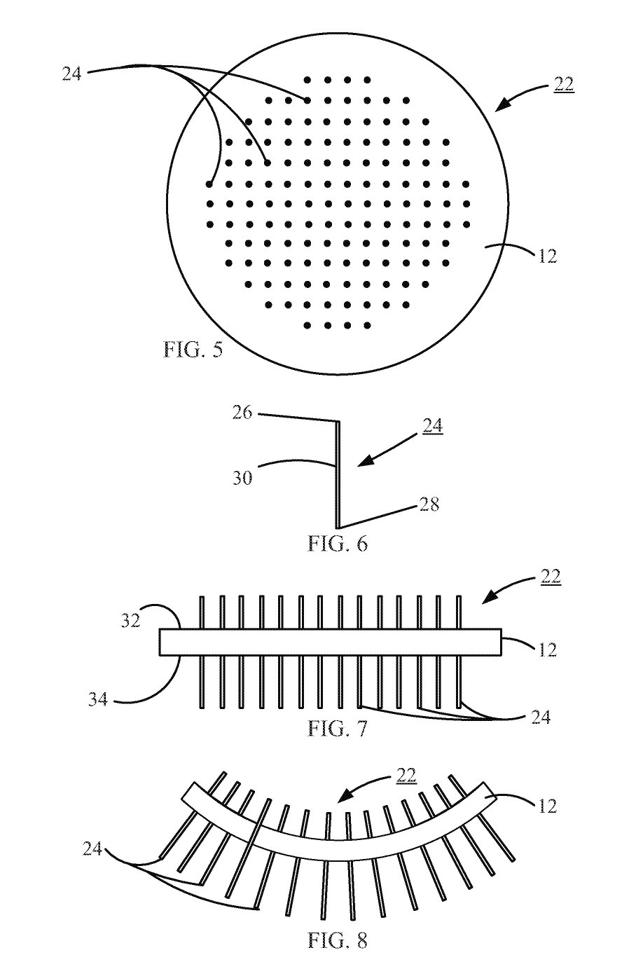

FIG. 5 shows an alternate embodiment of a sensor probe assembly 22, which is preferably formed from the conductive pin securement member 12, and a plurality of alternate preferred conductive pins 24. As shown by FIG. 6, preferably each alternate preferred conductive pin 24 includes a head portion 26, a tip portion 28, and a body portion 30, wherein the head portion 26 and the tip portion 28 have diameters substantially equal to the body portion 30. However, a skilled artisan will appreciate that conductive pins may have head, tip and body portion diameters different from one another. For example, the body portion may have a diameter greater than either the tip portion or head portion to accommodate insert molding of the conductive pins into a conductive pin securement member. It is further understood that the conductive pins may take on a profile that includes a bend in the body, tip, or head portions, as opposed to the cylindrical configuration of any suitable cross section geometric shape of the conductive pins shown by FIG. 2 and FIG. 6. It is still further understood, that the conductive pins may be formed by a plurality of individual components, including without limitation a spring, or may be formed from a coiled or other form of spring alone.

As with the preferred conductive pins 14, the alternate preferred conductive pins 24 are formed from a non-corrosive material, such as stainless steel, titanium, bronze, or a precious metal plating on a rigid substrate selected from a group including at least polymers and metals.

FIG. 7 shows the conductive pins 24 protruding through each the top and bottom surfaces, 32 and 34 respectfully, to accommodate improved conductivity of the alternate sensor probe assembly 22, with mating components. While FIG. 8 shows that the alternate sensor probe assembly 22 preferably retains the flexibility characteristics of sensor probe assembly 10 of FIG. 4.

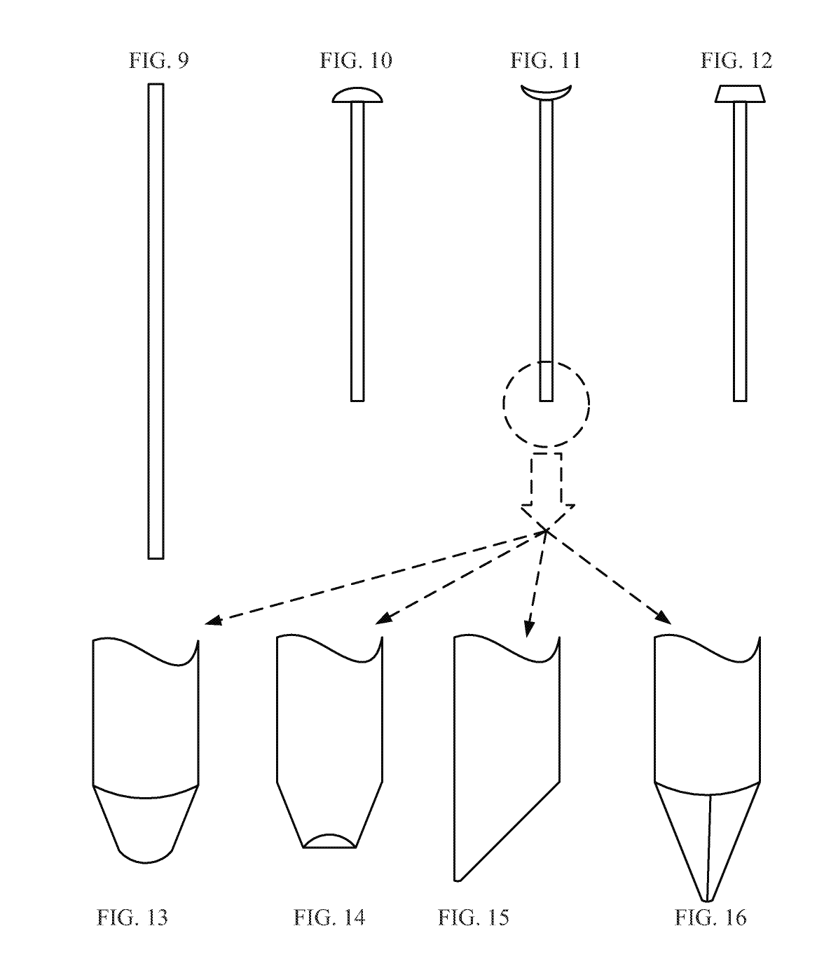

FIGS. 9, 10, 11, and 12 show just a few of a plurality of head configurations suitable for use on conductive pins. The particular configuration selected is a function of the device or component with which the conductive pins electrically cooperate. When a connector is used to interface with the sensor probe assembly, such as 10 or 22, the precise configuration will depend on the type and configuration of the pins associated with the connector, including whether the pins are male or female pins.

FIGS. 13, 14 (a cross section view), 15, and 16 show just a few of a plurality of tip configurations suitable for use on conductive pins. The particular configuration selected is a function of the materials used to form the conductive pins, and the environment in which the conductive pin will be placed. Examples of the use environment include where on the cranium the sensor will be placed, whether hair is present, and the sensitivity of the subject to the tips of the conductive pins.

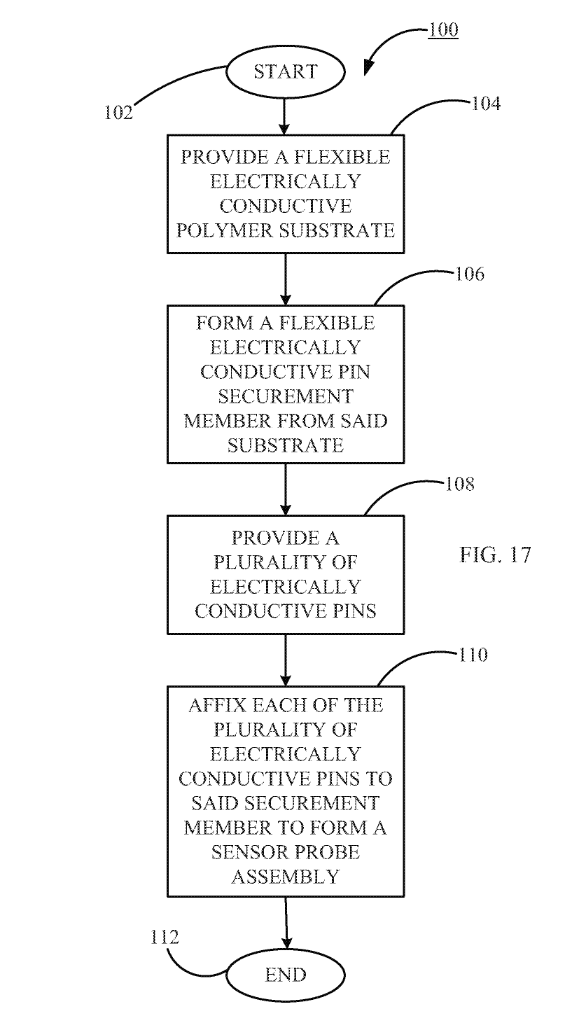

FIG. 17 shows a method 100, of making a sensor probe assembly, such as 10 or 22. The method begins at start step 102, and proceeds to process step 104, where a flexible conductive pin securement material is provided (also referred to herein as a flexible, electrically conductive, polymer substrate). At process step 106, a flexible, electrically conductive, pin securement member (such as 12) is formed from the flexible, electrically conductive, polymer substrate.

The process continues at process step 108, a plurality of electrically conductive pins (such as 14) is provided. At process step 110, each of the plurality of electrically conductive pins are affixed to the flexible, electrically conductive, pin securement member, and the process concludes at end process step 112 with the formation of a sensor probe assembly.

Turning to FIG. 18, shown therein is an embodiment of a novel, inventive, sensor assembly 200. Preferably, the sensor assembly 200 includes at least a sensor probe assembly 10, which provides a plurality of conductive pins 14, and a compressible electrically conductive member 202, in electrical communication with the sensor probe assembly. Preferably, the compressible electrically conductive member 202 is formed from a polyurethane polymer filled with conductive particles, which are preferably carbon particles. One such combination is a low density black conductive Polyurethane open cell flexible conductive foam material provided by Correct Products, Inc. of Richardson, Tex. However, as known in the art, conductive polymers may be formed from a plurality of polymer materials filled with conductive particles, the shape of which may be formed using well known manufacturing techniques that include at least molding, extrusion dies and sliced to thickness, formed in sheets and: die cut; cut with hot wire equipment; high pressure water jets, or steel rule dies.

As further shown by FIG. 18, the embodiment of the novel, inventive, sensor assembly 200 includes at least a signal processing circuit 204, in electrical communication with the compressible electrically conductive member 202, and a housing 206, confining the sensor probe assembly 10, the compressible electrically conductive member 202, and the signal processing circuit 204, to form the sensor assembly 200.

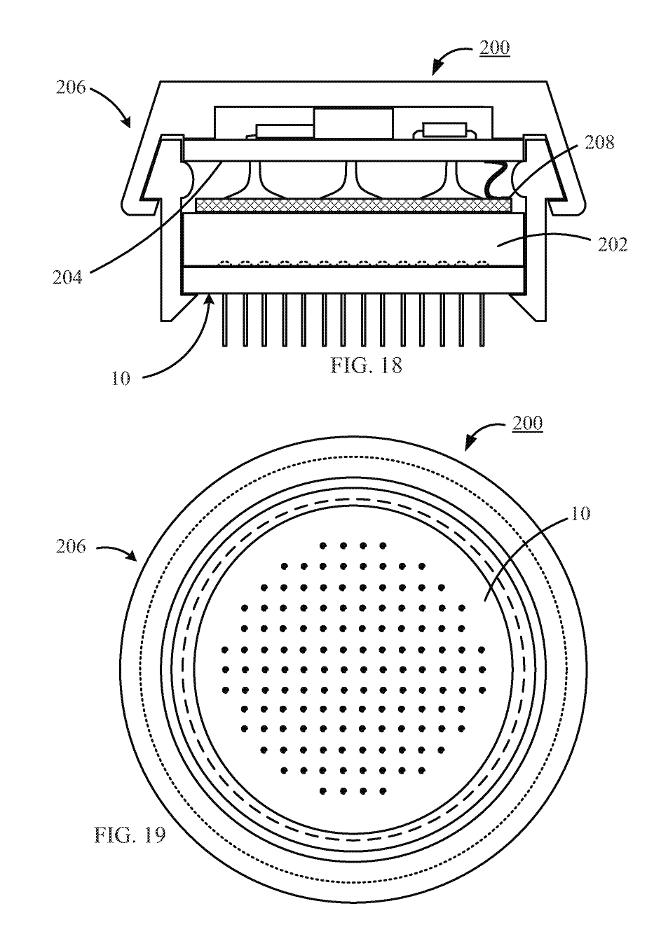

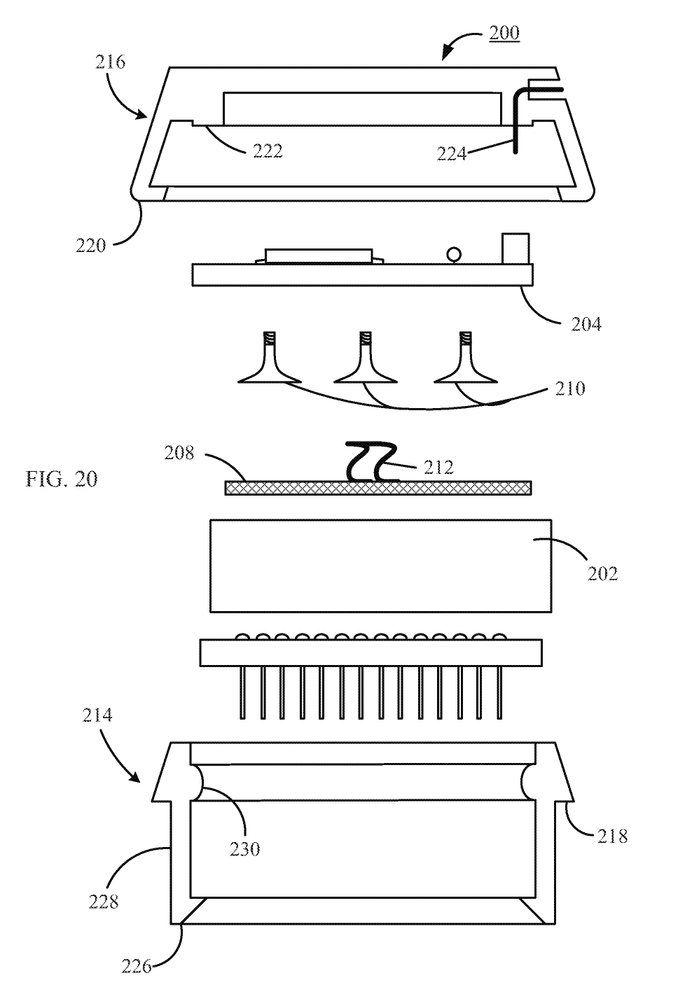

FIG. 19 shows the preferred embodiment of the sensor assembly 200 to be of a continuous curvilinear configuration; however, those skilled in the arts will recognize that any geometric shape may be presented by the sensor assembly 200. It is further noted that the sensor probe assembly 10, is confined by the housing 206 in such a manner that the sensor probe assembly 10, can be replaced without the disassembly of the entire sensor assembly 200.

The right side cross-section view and elevation of the preferred embodiment of the sensor assembly 200 of FIG. 20, reveals a rigid conductive member 208, and a plurality of standoffs 210, disposed between the signal processing circuit 204, and the electrically conductive member 202 (shown in its decompressed form). Preferably, the rigid conductive member 208 is in electrical interaction with a signal conductor 212, and the signal conductor 212 is in electrical communication with signal processing circuit 204. The standoffs 210 are preferably attached to the signal processing circuit 204, and functions to provide a slight compressive load on the compressible electrically conductive member 202. The compressive load allows for decompression of the compressible electrically conductive member 202 while the probe assembly is being exchanged. This particular feature promotes stability of the rest of components within the housing 206, when the sensor probe assembly is absent from the remaining components of the sensor assembly 200.

As is further shown by FIG. 20, the housing 206, of FIG. 18, preferably includes a component chamber 214, and a confinement cover 216. The component chamber 214 preferably includes a confinement cover retention feature 218, which interacts with a retention member 220 of the confinement cover 216. In a preferred embodiment, the confinement cover 216 “snaps” onto the component chamber 214. In a preferred embodiment, the component chamber 214 and the confinement cover 216 are formed from a shape retaining material that provides sufficient flexibility to allow the retention member 220 of the confinement cover 216 to pass by the confinement cover retention feature 218 of the component chamber 214, and then lock together the confinement cover 216 with the component chamber 214. As those skilled in the art will recognize that there are a number of engineering materials suitable for this purpose including, but not limited to, metals, polymers, carbon fiber materials, and laminates.

In the preferred embodiment of the sensor assembly 200, the confinement cover 216 further includes at least a signal processing circuit retention feature 222 and a connector pin 224 supported by the signal processing circuit retention feature 222, while the component chamber 214 further includes at least: a sensor probe assembly retention feature 226; a side wall 228 disposed between the confinement cover retention feature 218 and the sensor probe assembly retention feature 226; and a holding feature 230 provided by the side wall 228 and adjacent in the confinement cover retention feature 218.

In the preferred embodiment of the sensor assembly 200, the compressibility of the compressible electrically conductive member 202 promotes an ability to change out the sensor probe assembly 10, without disturbing the interaction of the signal processing circuit 204 and the rigid conductive member 208, or to change out the processing circuit 204 and the rigid conductive member 208 without disturbing the sensor probe assembly 10. When the sensor probe assembly 10 is removed from the preferred embodiment of the sensor assembly 200, the compressible electrically conductive member 202 explains to interact with the sensor probe assembly retention feature 226 thus maintaining the rigid conductive number 208 in pressing contact with standoffs 210. When the signal processing circuit 204, standoffs 210, and the rigid conductive member 208 are removed from the preferred embodiment of the sensor assembly 200, the compressible electrically conductive member 202 explains to interact with the holding feature 230 to preclude the inadvertent removal of the sensor probe assembly 10 from communication with the sensor probes assembly retention feature 226.

As will be recognized by skilled artisans, it is the collaborative effect of the pin or pins 14 of the sensor probe assembly 10 interacting with the cranium of the subject that promotes transference of brainwave signals of the subject to the signal processing circuit 204. To promote the conveyance of the brainwave signal, the sensor probe assembly 10 further provides a conductive pin securement member 12 cooperating in retention contact with the plurality of conductive pins 14.

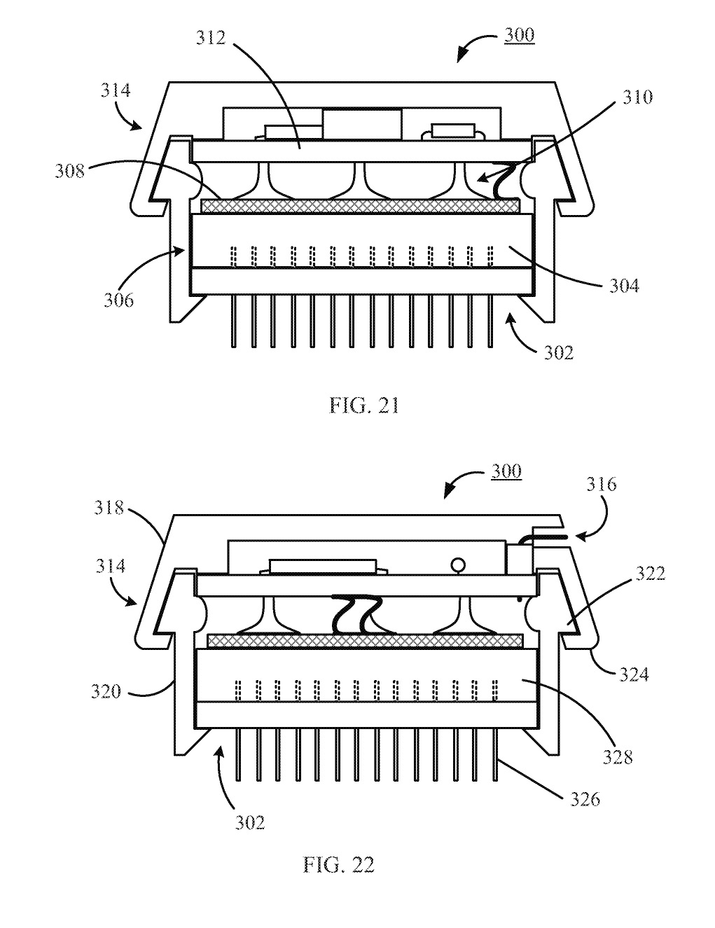

FIG. 21 shows an alternate preferred embodiment of a novel, inventive, standalone sensor assembly 300. Preferably, the standalone sensor assembly 300 includes at least an electrically conductive member 302 forming a first plate 304 of a capacitor 306, a dielectric material 308, adjacent the first plate 304, a second plate 310 of the capacitor 306 communicating with the dielectric material 308, and a signal processing circuit 312 in electrical communication with said dielectric material 308. FIG. 21 further shows a housing 314 confining the first plate 304 of the capacitor 306, the dielectric material 308, the second plate 310, and the signal processing circuit 312 to form the standalone sensor assembly 300.

FIG. 22 shows the standalone sensor assembly 300 further includes a communication port 316, useful for transferring processed signals to an external system for analysis, and that the housing 314 preferably includes a component chamber 318, and a confinement cover 320. The component chamber 318 preferably includes a confinement cover retention feature 322, which interacts with a retention member 324 of the confinement cover 320. In a preferred embodiment, the confinement cover 320 “snaps” onto the component chamber 318.

In a preferred embodiment, the component chamber 318 and the confinement cover 320 are formed from a shape retaining material that provides sufficient flexibility to allow the retention member 324 of the confinement cover 320 to pass by the confinement cover retention feature 322 of the component chamber 318, and then lock together the confinement cover 320 with the component chamber 318. As those skilled in the art will recognize that there are a number of engineering materials suitable for this purpose including, but not limited to, metals, polymers, carbon fiber materials, and laminates.

In a preferred embodiment, the electrically conductive member 302 forming the first plate 304 of the capacitor 306 includes at least, but is not limited to, a plurality of at least partially insulated pins 326, communicating with a conductive member 328, wherein the conductive member is in direct contact adjacency with the dielectric material 308. In operation, the voltage potential is present between the first plate 304 and the second plate 310, which results in a charge build up on the dielectric material 308, and it is the level of the charge build up that is processed by the signal processing circuit 312. The plurality of at least partially insulated pins 326, each preferably have four degrees of freedom i.e.: yaw; pitch; roll; and z axis. The multiple degrees of freedom accommodates the topography differences in the cranium of different subjects, to promote a subject adaptable, alternate preferred embodiment of the novel, inventive, standalone sensor assembly 300.

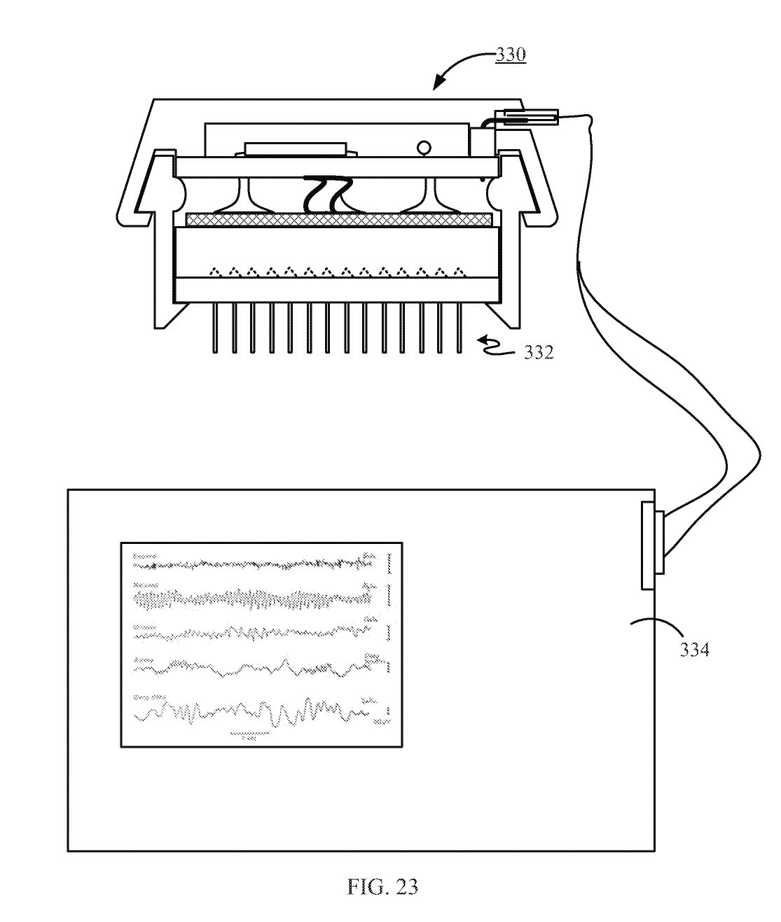

FIG. 23 shows an alternative preferred embodiment of the novel, inventive, standalone sensor assembly 330, having a plurality of alternate conductive pins 332; however, the remaining components are substantially equal to the corresponding remaining components of the preferred embodiment of the novel, inventive, standalone sensor assembly 200. Further shown by FIG. 23, is a brainwave processing system 334, which may be, for example, an Electroencephalography (EEG) 334.

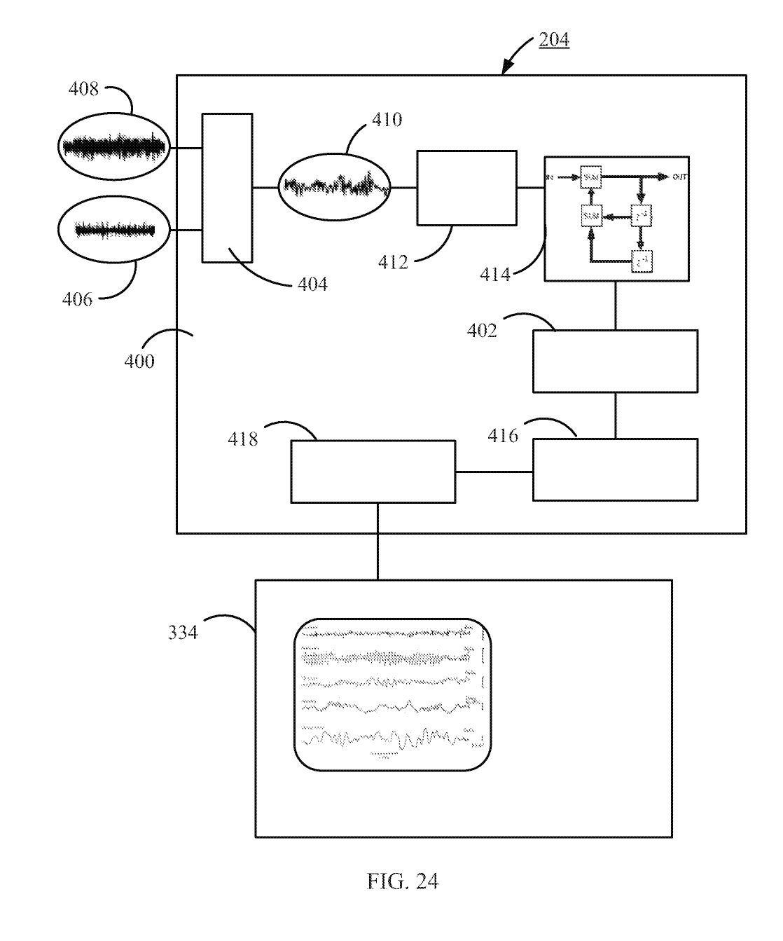

As is shown by FIG. 24, a preferred embodiment of the signal processing circuit 204 includes at least, but is not limited to, a printed circuit member 400, and a processor 402, interacting with said printed circuit member 400, the processor 402 receiving signals from a sensor probe assembly, such as 200 of FIG. 18, and communicating the signals to a brainwave processing system, such as 334 of FIG. 23.

The preferred embodiment of the signal processing circuit 204 further includes at least, but is not limited to, a differential amplifier 404, interacting with the printed circuit member 400, a reference signal 406 communicating with the differential amplifier 404, and a subject signal 408 provided by a sensor probe assembly, such as 200 of FIG. 18, when the sensor probe assembly 200 is in electrical contact with a cranium of a subject. Preferably, the differential amplifier 404 compares the reference signal 406 to the subject signal 408 and discards common signal patterns presented by said reference and subject signals, 404 and 406, to provide a native brainwave signal 410, of the subject.

Further, the preferred embodiment of the signal processing circuit 204 includes at least, but is not limited to, an analog to digital converter with a digital signal processing core 412, interacting with the differential amplifier 404 and processing the native brainwave signal 410, provided by the differential amplifier 404, and outputting a digital signal representative of the native brainwave signal, and an infinite impulse response filter 414, interacting with the analog to digital converter 412, to serve as a band pass filter for said digital signal.

Still further, the preferred embodiment of the signal processing circuit 204 shown in FIG. 24, includes at least, but is not limited to, a memory 416, also referred to herein as a buffer 416, communicating with the processor 402, and storing processed native brainwave signals, and a communication port 418 communicating with the buffer 416, the communication port is preferably responsive to the processor 402 for communicating processed native brainwave signals to the brainwave processing system 334.

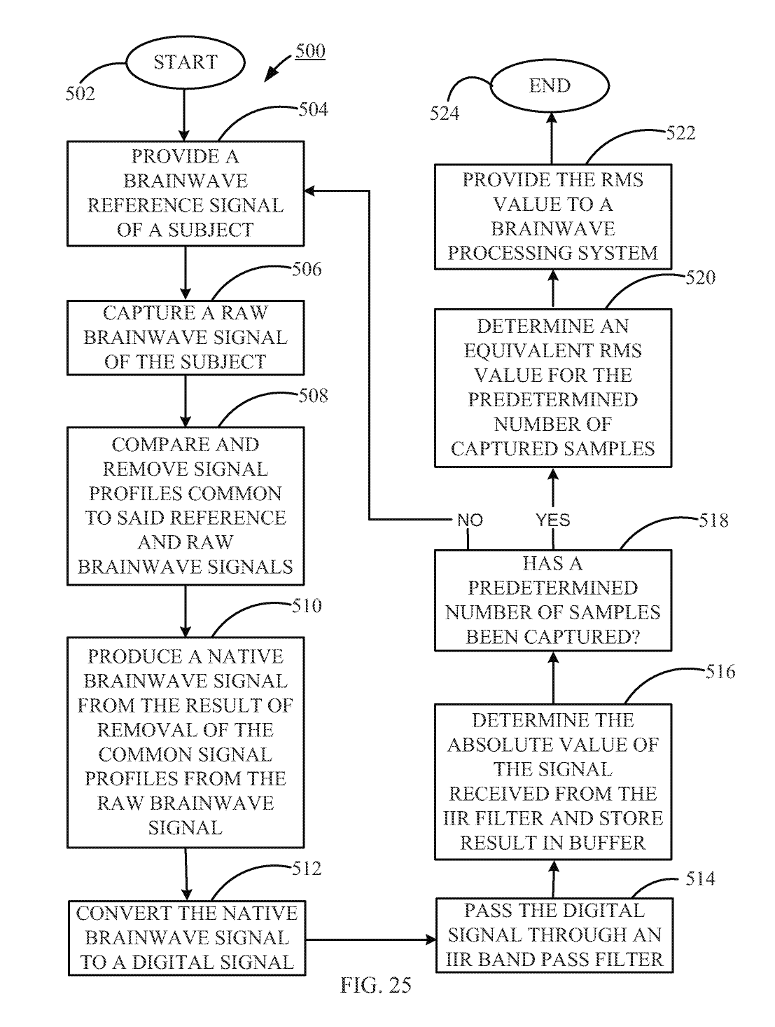

FIG. 25 shows a method 500, of using a signal processing circuit, such as 400, of FIG. 24. The method begins at start step 502, and proceeds to process step 504, where a brainwave reference signal (such as 406) of a subject is provided. At process step 506, a raw brainwave signal (such as 408) of the subject is captured. At process step 508, the signal profiles of the reference and raw brainwave signals are compared, and signal profiles common to both are removed, and at process step 510, a native brainwave signal (such as 410) is produced from the result of the removal of signal profiles common to both the reference and raw brainwave signals.

The process continues at process step 512, where the native brainwave signal is converted to a digital band of frequency signal, and passed to an IIR band pass filter (such as 414) at process step 514. At process step 516, an absolute value of the digitized signal received from the IRR filter is determined by a processor (such as 402). It is noted that in a preferred embodiment the IIR filter is programmable and responsive to the processor, and that multiple IIR filters may be employed to capture a multitude of discrete band frequencies (typically having about a 5 Hz spread, such as 10 to 15 Hz out of a signal having a frequency range of about 0.5 Hz to 45 Hz)), or the programmable IIR filter may be programed to collect a certain number of discrete, common frequency band samples, each sample obtained over a predetermined amount of time, and then reprogramed to obtain a number of different, discrete, common frequency band samples.

The process continues at process step 518, where the processor determines if a predetermined number of samples of the absolute value each discrete band frequency of interest has been stored in a buffer (such as 416). If the number of captured desired samples has not been met, the process reverts to process step 504. If the number of captured desired samples has been met, the process proceeds to process step 520. At process step 520, the processor determines an equivalent RMS (root mean square) value for each of the plurality of discrete band frequency, absolute value sets of samples, and those values are provided to a brainwave processing system (such as 334) at process step 522. At process step 524, the process ends.

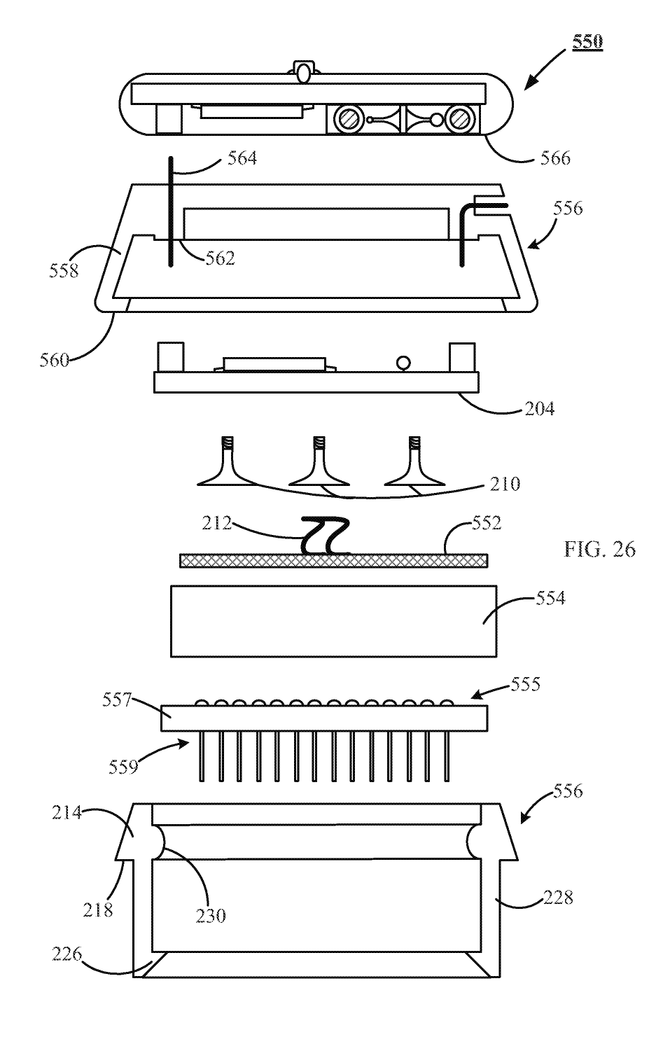

The right side cross-section view in elevation of the preferred embodiment of the sensor assembly 550 of FIG. 26 reveals an electrical element 552, and a plurality of standoffs 210, disposed between the signal processing circuit 204, and an electrically sympathetic member 554. Preferably, the electrical element 552 is a rigid conductive member 552 in electrical interaction with the signal conductor 212, and the signal conductor 212 is in electrical communication with the signal processing circuit 204. In one preferred embodiment, the electrically sympathetic member 554 is a compressible electrically conductive member 554, and the standoffs 210, are preferably attached to the signal processing circuit 204, and functions to provide a slight compressive load on the compressible electrically conductive member 554. The compressive load allows for decompression of the compressible electrically conductive member 554 while the probe assembly 555 is being exchanged. This particular feature promotes stability of the rest of components within a housing 556, when the sensor probe assembly is absent from the remaining components of the sensor assembly 550.

As is further shown by FIG. 26, the housing 556, preferably includes the component chamber 214, and a confinement cover 558. The component chamber 214 preferably includes a confinement cover retention feature 218, which interacts with a retention member 560 of the confinement cover 558. In a preferred embodiment, the confinement cover 558 “snaps” onto the component chamber 214. In a preferred embodiment, the component chamber 214 and the confinement cover 558 are formed from a shape retaining material that provides sufficient flexibility to allow the retention member 560 of the confinement cover 558 to pass by the confinement cover retention feature 218 of the component chamber 214, and then lock together the confinement cover 558 with the component chamber 214. As those skilled in the art will recognize that there are a number of engineering materials suitable for this purpose including, but not limited to, metals, polymers, carbon fiber materials, and laminates.

In the preferred embodiment of the sensor assembly 550, the confinement cover 558 further includes at least a signal processing circuit retention feature 562, the connector pin 564 supported by the signal processing circuit retention feature 562, and an oscillation device conductor 564, while the component chamber 214 further includes at least: a sensor probe assembly retention feature 226; a side wall 228 disposed between the confinement cover retention feature 218 and the sensor probe assembly retention feature 226; and a holding feature 230 provided by the side wall 228 and adjacent in the confinement cover retention feature 218. Preferably, the oscillation device conductor 564 passes signals between the signal processing circuit 204 and an oscillation device 566, which is responsive to the signal processing circuit 204.

In the preferred embodiment of the sensor assembly 550, the compressibility of the compressible electrically conductive member 202 promotes an ability to change out the sensor probe assembly 555, without disturbing the interaction of the signal processing circuit 204 and the rigid conductive member 552, or to change out the processing circuit 204 and the rigid conductive member 552 without disturbing the sensor probe assembly 555. When the sensor probe assembly 555 is removed from the preferred embodiment of the sensor assembly 550, the compressible electrically conductive member 554 explains to interact with the sensor probe assembly retention feature 226 thus maintaining the rigid conductive number 208 in pressing contact with the standoffs 210. When the signal processing circuit 204, standoffs 210, and the rigid conductive member 552 are removed from the preferred embodiment of the sensor assembly 550, the compressible electrically conductive member 554 explains to interact with the holding feature 230 to preclude the inadvertent removal of the sensor probe assembly 555 from communication with the sensor probes assembly retention feature 226.

To promote the conveyance of the brainwave signal, the sensor probe assembly 555 further provides a conductive securement member 557 cooperating in retention contact with an electrically conductive surface 559, which in one preferred embodiment is a plurality of electrically conductive surfaces 559. In one embodiment, as will be recognized by skilled artisans, it is the collaborative effect of plurality of electrically conductive surfaces 559 of the sensor probe assembly 555 interacting with the cranium of the subject that promotes transference of brainwave signals of the subject to the signal processing circuit 204.

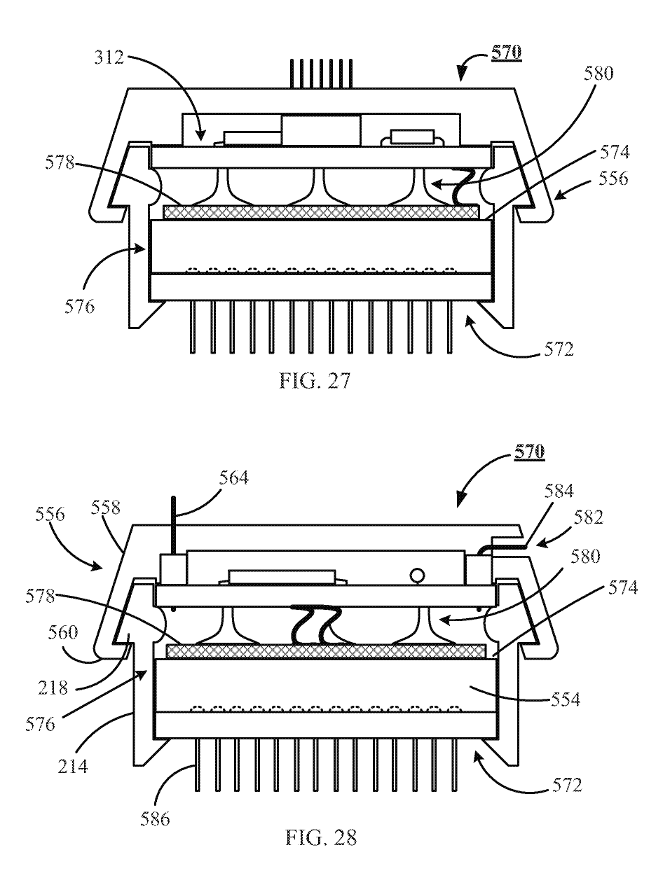

FIG. 27 shows an alternate preferred embodiment of a novel, inventive, standalone sensor assembly 570. Preferably, the standalone sensor assembly 570 includes at least an electrically conductive member 572 forming a first plate 574 of a capacitor 576, an electrically responsive member 578, which in a preferred embodiment is a dielectric material 578, adjacent the first plate 574, a second plate 580 of the capacitor 576 communicating with the dielectric material 578, and a signal processing circuit 312 in electrical communication with said dielectric material 578. FIG. 27 further shows a housing 556 confining the first plate 574 of the capacitor 576, the dielectric material 578, the second plate 580, and the signal processing circuit 312 to form the standalone sensor assembly 570.

FIG. 28 shows the standalone sensor assembly 570 further includes a communication port 582, useful for transferring processed signals to an external system for analysis, and that the housing 556 preferably includes a component chamber 214, and a confinement cover 558. The component chamber 214 preferably includes a confinement cover retention feature 218, which interacts with a retention member 560 of the confinement cover 558. In a preferred embodiment, the confinement cover 558 “snaps” onto the component chamber 214, and a signal conductor 584 of the communication port 582, and the oscillation device conductor 564 each make an electrical connection with the signal processing circuit 312.

In a preferred embodiment, the electrically conductive member 572 forming the first plate 574 of the capacitor 576 includes at least, but is not limited to, a plurality of at least partially insulated pins 586, communicating with a conductive member 554, wherein the conductive member is in direct contact adjacency with the dielectric material 578. In operation, the voltage potential is present between the first plate 574 and the second plate 580, which results in a charge build up on the dielectric material 578, and it is the level of the charge build up that is processed by the signal processing circuit 312. The plurality of at least partially insulated pins 586, each preferably have four degrees of freedom i.e.: yaw; pitch; roll; and z axis. The multiple degrees of freedom accommodates the topography differences in the cranium of different subjects, to promote a subject adaptable, alternate preferred embodiment of the novel, inventive, standalone sensor assembly 570.

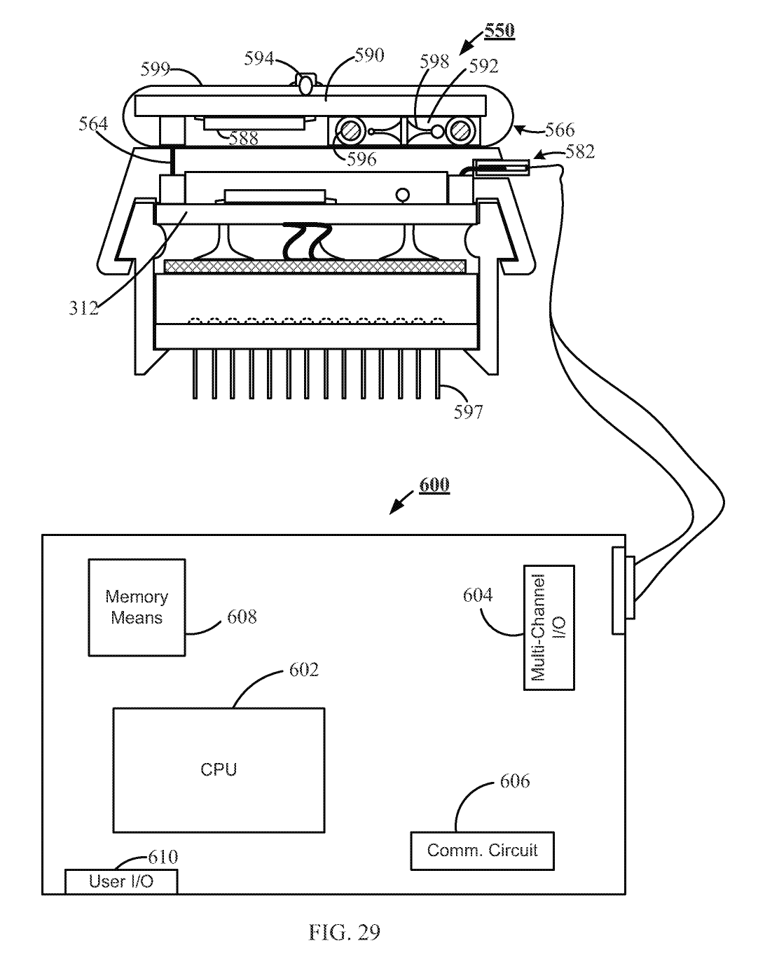

In a preferred embodiment illustrated by FIG. 29, the preferred oscillation device 566 is shown to include at least, but not limited to, an oscillation device controller 588, responsive to the signal processing circuit 312, the oscillation device controller 588 is preferably in electrical communication with an electrical support member 590. The preferred oscillation device 566 further preferably includes a vibration inducing member 592 responsive to the oscillation device controller 588, and a status indicator 594 in electrical communication with and responsive to the signal processing circuit 312. Preferably the vibration inducing member 592 provides a stator 596 responsive to the oscillation device controller 588, and an out of balance rotor 598 responsive to the stator 596. Preferably, the preferred oscillation device 566 also includes a tactile housing 599, confining the vibration inducing member 592, the oscillation device controller 588, and said status indicator 594. In one embodiment the status indicator 594 is an LED, which blinks in unison with captured brainwave activity. In another embodiment, the preferred oscillation device 566 is responsive to a condition of the conductive pins 597 interfacing with the scalp of a subject presents a circuit with an excessive level of impedance. Activation of the preferred oscillation device 566 promotes the breakthrough of the high resistance epidermal layer of skin on the scalp of the subject and improved electrical contact. In another embodiment, the preferred oscillation device 566 provides tactile feedback to the subject to provide awareness of a particular brain state of interest to the subject.

FIG. 29 further shows a preferred brainwave processing system 600, which includes at least, but not limited to, a central processing unit (“CPU”) 602 that communicates with the sensor assembly 550 through a multi-channel input/output (“I/O”) circuit 604. The CPU 602 further electronically interacts with a communication control circuit 606, which accommodates communication with remote devices, including, but not limited to wireless communications. In a preferred embodiment, the preferred brainwave processing system 600 further provides a memory means 608, which may be either a memory (either volatile or non-volatile), or a storage device such as, but not limited to, a flash memory, a solid state disc drive, or a disc drive, or which may be incorporated into the CPU 602. In any case, the memory means 608 facilitates storage of an operating code purposefully written to control the operations of the sensor assembly 550.

The memory means 608 further preferably stores mental exercise routines for a subject, which can be called upon by the CPU 602 in response to a brain state of the subject different than a desired brain state of the subject. Preferably, when a particular, desired brain state of the subject is not shown to be present, the CPU 602 preferably selects a mental or physiological exercise to be performed by the subject. The CPU 602 may direct the agitation of the subject cranium by signaling the activation of the oscillation device 566 of the sensor assembly 550, which provides an alert to the subject to, for example without limitation, commence with a breathing exercise.

Alternatively, for example without limitation, the CPU 602 may communicate through a multi-functional user interface 610 to download commands to an external device, such as but not limited to: an MP3 player; a smart phone; tablet; or a video game delivery device, which presents the selected exercise to the subject. The CPU 602 preferably further processes performance data received from the sensor assembly 550, and stores the processed performance data (either physiological or neurological) in the storage means 608 for delivery to an external database upon a request from said database for said stored performance data.

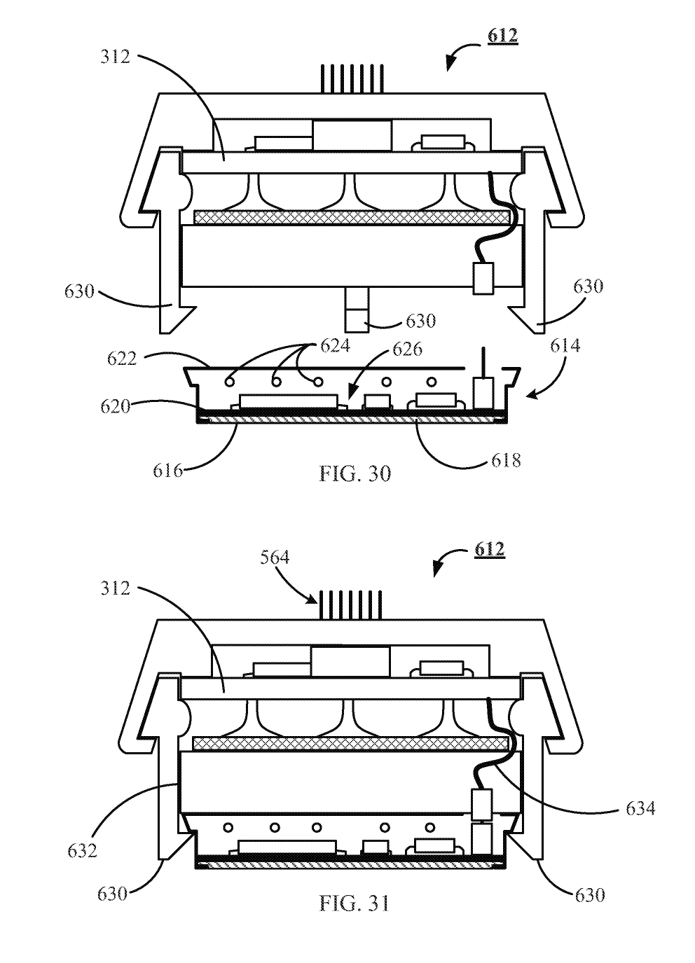

FIGS. 30 and 31 provide an alternate alternative preferred embodiment of a capacitance sensor assembly 612, which includes a capacitance probe assembly 614, communicating with the signal processing circuit 312. Preferably, the capacitance probe assembly 614 includes a first conductor 616 in direct electrical contact with a dielectric material 618, and a second conductor 620 in direct electrical contact with the dielectric material 618. The capacitance probe assembly 614 further preferably includes a capacitance probe shield 622, which provides a plurality of vent apertures 624 that assist in modulating the thermal environment surrounding a capacitance signal processing circuit 626.

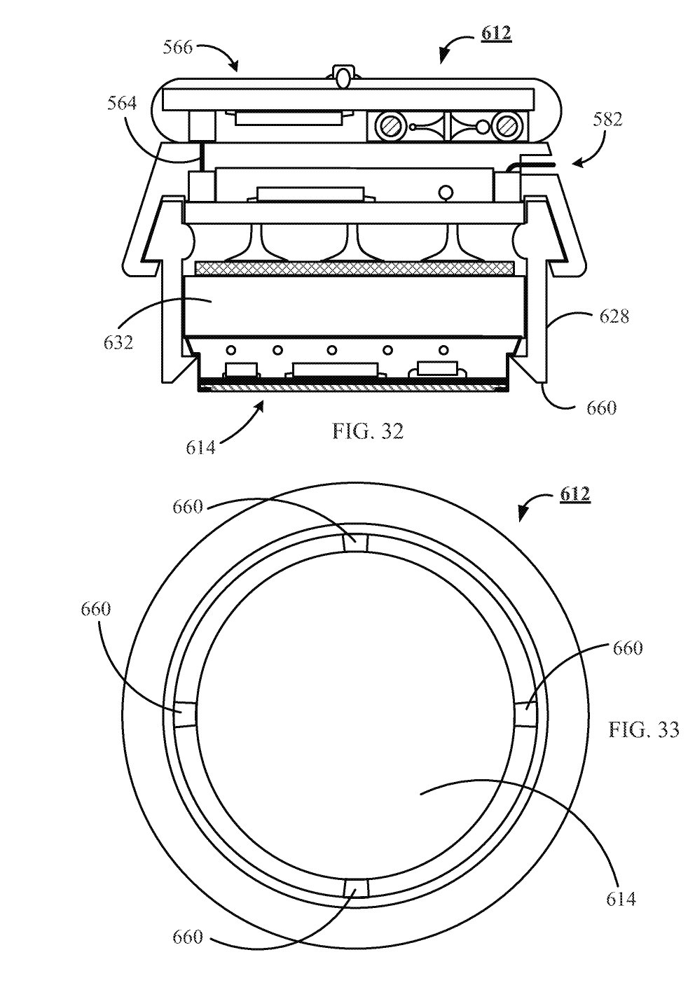

FIG. 31 shows the capacitance sensor assembly 612 preferably includes the oscillation device conductor 564, which preferably passes signals between the signal processing circuit 312 and the oscillation device 566 of FIG. 32, as well as the communication port 582, useful for transferring processed signals to a brainwave processing system (such as 600 of FIG. 29) for analysis.

In a preferred embodiment, a component chamber 628, similar in function to the component chamber 214 of FIG. 28, provides a plurality of attachment tangs 630 used to secure the capacitance probe assembly 614 firmly positioned within the component chamber 628 of the capacitance sensor assembly 612, as shown by FIG. 33. In one embodiment of the capacitance sensor assembly 612, the capacitance probe assembly 614 is offset from the signal processing circuit 312 by a compressible member 632, and communicated with the signal processing circuit 312 via an electrical connection assembly 634 of FIG. 31.

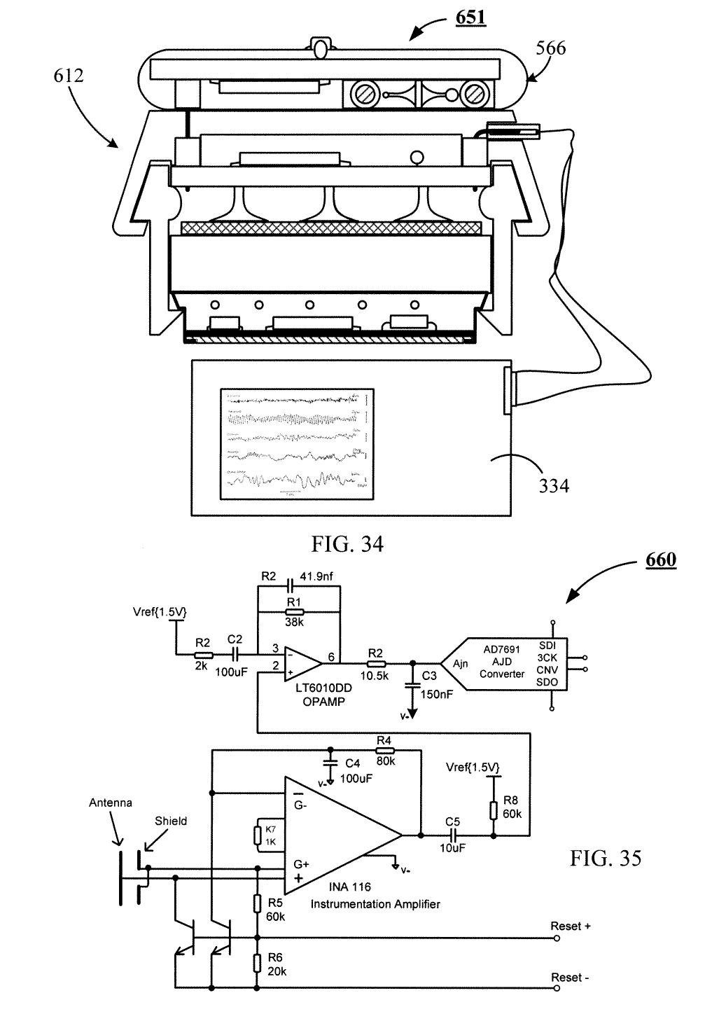

FIG. 34 shows the capacitance sensor assembly 612 configured with the oscillation device 566, and communicating with the brainwave processing system 334, which may be, for example, an Electroencephalography (EEG) 334, or in the alternative the preferred brainwave processing system 600. Collectively, the capacitance sensor assembly 612 configured with the oscillation device 566 forms a preferred capacitance sensor 651.

An exemplary circuit of the capacitance signal processing circuit 626, of the capacitance probe assembly 614 that senses, amplifies and acquires the raw brainwave signal 408 (of FIG. 24), is shown in FIG. 35. Preferably, the signal on the skin of a subject capacitively couples to a first conductor of a capacitance element. Preferably, the capacitance element further includes at least, but is not limited to: a dielectric material in pressing contact with the first conductor; and a second conductor in pressing contact with and separated from the first conductor by the dielectric material. Preferably, the capacitance element provides the acquired brainwave signal 408 to the signal processing circuit 312 (of FIG. 21).

In a preferred embodiment, amplification of the raw brainwave signal 408 is accomplished by an instrumentation amplifier, such as the INA116 provided by Texas Instruments, Inc. of Dallas Tex., is preferably configured for a gain of 50. This component has been seen to have an extremely low input bias current of 3 fA (typical) and an input current noise of 0.1 fA/√Hz (typical). It also features guard pin outputs, which follow the positive and negative inputs with a gain of 1. Preferably, in addition to using the positive guard to support a guard ring around the positive input pin, it is also used to drive a shielding metal plate that minimizes electric field pick up from sources other than the scalp. This shield is preferably implemented as an inner layer of metal on the printed circuit above the sensor metal layer. As those skilled in the art will recognize, because it is actively driven to duplicate the input voltage, it avoids parasitic capacitance division of signal gain.

Although the input bias current is extremely small, it has been noted that if left unattended, it will drive the high-impedance positive input node of the amplifier toward one of the supply rails. A means of combating this is a preferred use of a reset circuit which includes two transistors and two resistors. Preferably, the transistors are turned on by an external circuit when the input voltage nears the common-mode input range of the amplifier. When not driven, the base and emitter nodes of the transistors are pulled up by the guard output. Preferably, this is done to minimize leakage currents (and especially the resultant current noise) from the transistors. In a preferred embodiment, the negative amplifier input is made to track the slowly changing positive input with the feedback loop consisting of R4 and C4. In a preferred embodiment, this loop also serves to cut off input signals of frequencies below about 1 Hz.

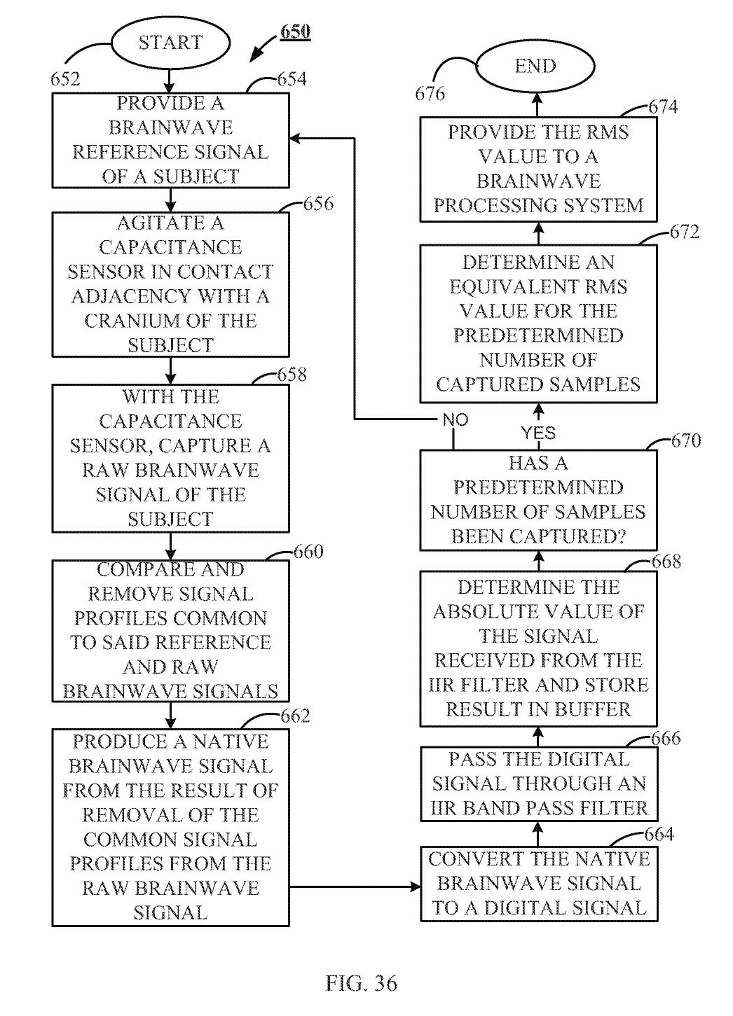

FIG. 36 shows a method 650, of using a signal processing circuit, such as 312, of FIG. 27. The process begins at start step 652, and proceeds to process step 654, where a brainwave reference signal (such as 406) of a subject is provided. At process step 656, a capacitance sensor (such as 612 configured with an oscillation device such as 566) in contact adjacency with a cranium of the subject is agitated. At process step 658, a raw brainwave signal (such as 408) of the subject is captured using the capacitance sensor. At process step 660, the signal profiles of the reference and raw brainwave signals are compared, and signal profiles common to both are removed, and at process step 662, a native brainwave signal (such as 410) is produced from the result of the removal of signal profiles common to both the reference and raw brainwave signals.

The process continues at process step 664, the native brainwave signal is converted to a digital band of frequency signal, and passed to an IIR band pass filter (such as 414) at process step 666. At process step 668, an absolute value of the digitized signal received from the IIR filter is determined by a processor (such as 402). It is noted that in a preferred embodiment the IIR filter is programmable and responsive to the processor, and that multiple IIR filters may be employed to capture a multitude of discrete band frequencies (typically having about a 5 Hz spread, such as 10 to 15 Hz out of a signal having a frequency range of about 0.5 Hz to 45 Hz), or the programmable IIR filter may be programed to collect a certain number of discrete, common frequency band samples, each sample obtained over a predetermined amount of time, and then reprogramed to obtain a number of different, discrete, common frequency band samples.

The process continues at process step 670, the processor determines if a predetermined number of samples of the absolute value each discrete band frequency of interest has been stored in a memory means (such as 608). If the number of captured desired samples has not been met, the process reverts to process step 654. If the number of captured desired samples has been met, the process proceeds to process step 672. At process step 672, the processor determines an equivalent RMS (root mean square) value for each of the plurality of discrete band frequency, absolute value sets of samples, and those values are provided to a brainwave processing system (such as 334) at process step 674. At process step 676, the process ends.

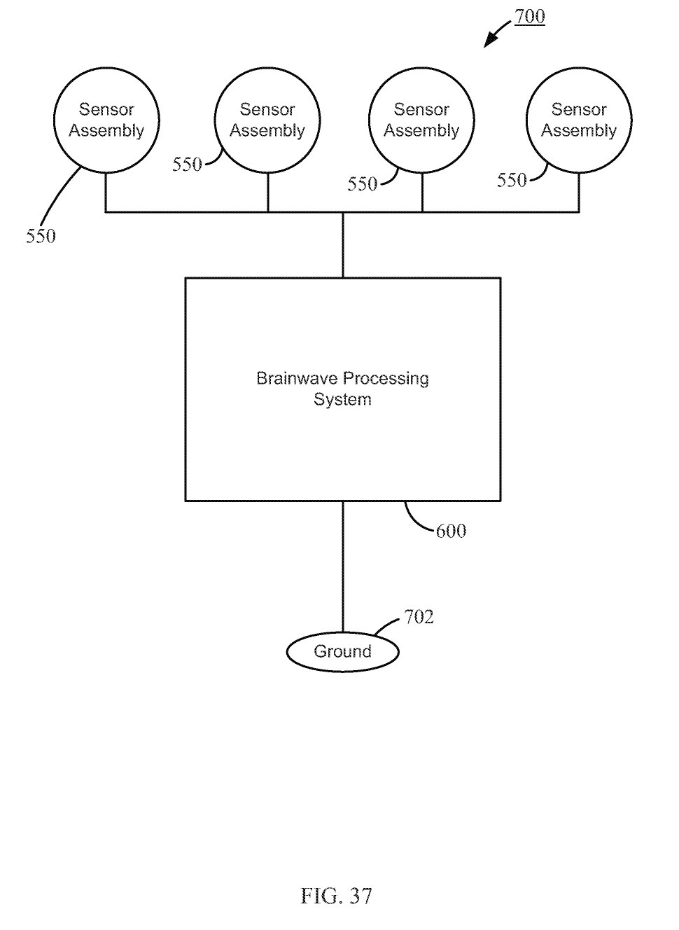

FIG. 37 shows a first embodiment of an inventive standalone neurophysiologic performance measurement and training system 700, which preferably includes at least four dry sensor assemblies 550 electrically connected to the preferred brainwave processing system 600, and a ground reference 702 electrically interacting with the preferred brainwave processing system 600.

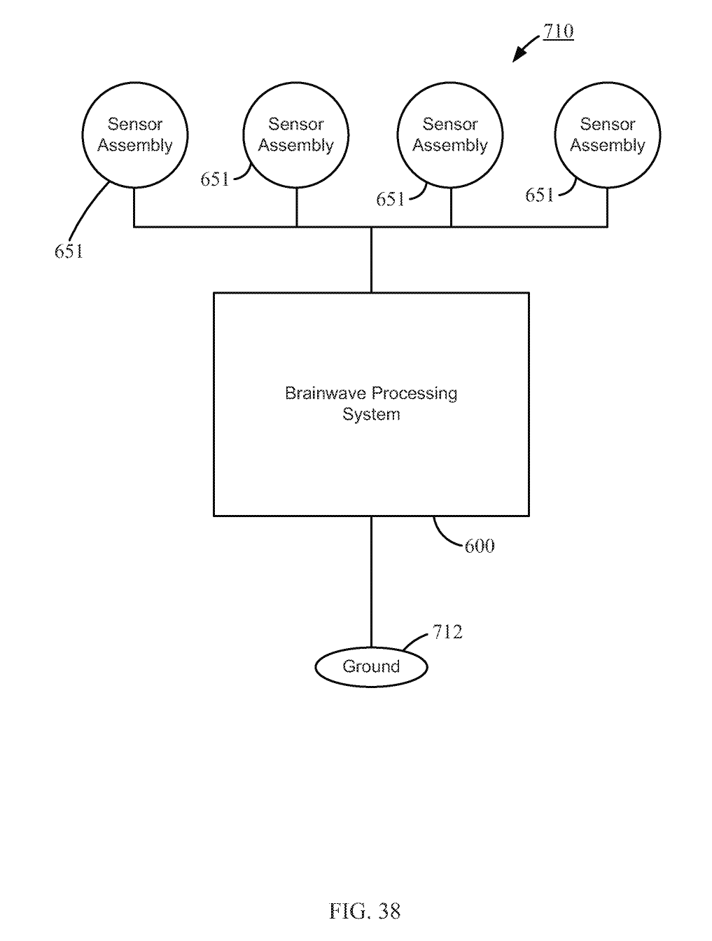

FIG. 38 shows a second embodiment of an inventive standalone neurophysiologic performance measurement and training system 710, which preferably includes at least four capacitance sensor assemblies 651 electrically connected to the preferred brainwave processing system 600, and a ground reference 712 electrically interacting with the preferred brainwave processing system 600.

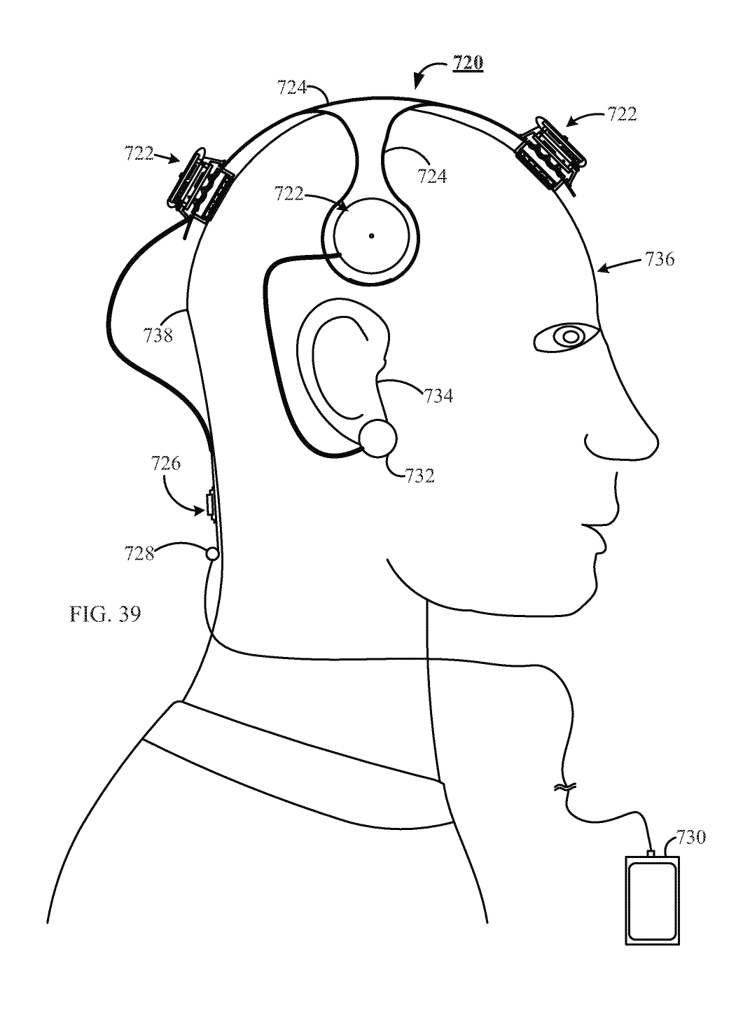

FIG. 39 shows a preferred configuration of an inventive standalone neurophysiologic performance measurement and training system 720, which preferably includes at least four sensor assemblies 722 supported by a sensor assembly web 724, a preferred brainwave processing system 726 that includes a multi-channel user interface 728 electrically interacting with an electronic device 730, and a ground reference 732 interacting with an ear 734 of a subject 736 and electrically interacting with the preferred brainwave processing system 726. Preferably, the sensor web assembly is formed to support each of the sensor assemblies 722, provide a communication buss between the brainwave processing system 726 and each of the sensor assemblies 722 and the ground reference 732, and facilitate a pressing contact interface between each of the sensor assemblies 722 and a cranium 738 of the subject 736. Preferably, the sensor assemblies 722 may be if any type of neurophysiologic monitoring sensor including, but not limited to, the dry sensor assembly 550, or the capacitance probe sensor 651.

As will be apparent to those skilled in the art, a number of modifications could be made to the preferred embodiments which would not depart from the spirit or the scope of the present invention. While the presently preferred embodiments have been described for purposes of this disclosure, numerous changes and modifications will be apparent to those skilled in the art. Insofar as these changes and modifications are within the purview of the appended claims, they are to be considered as part of the present invention.

What is claimed is:

1. A device comprising:
a plurality of sensor assemblies, each providing at least one electrically responsive surface;
an oscillation device communicating with said sensor assembly, wherein said sensor assembly includes at least a signal processing circuit in electrical communication with the oscillation device to selectively agitate said at least one electrically responsive surface;
a brainwave processing system communicating with each of the plurality of sensor assemblies; and
a ground reference interacting with the brainwave processing system, wherein a selected one of the plurality of sensor assemblies provides a reference signal for each of the remaining sensor assemblies, and in which each electrically responsive surface communicating with a cranium of a subject.

2. The device of claim 1, in which each said sensor assembly further comprising:
an electrically sympathetic member in electrical communication with said at least one electrically responsive surface,
an electrical element in electrical communication with said electrically sympathetic member; and
a signal conductor interacting with said electrical element and communicating signals facilitated by said at least one electrically responsive surface to said signal processing circuit.

3. The device of claim 2, in which said sensor assembly further comprising a housing confining said at least one electrically responsive surface, said electrically sympathetic member, said electrical element, said signal conductor and said signal processing circuit to collectively form said sensor assembly.

4. The device of claim 1, in which said oscillation device comprising:
an oscillation device controller responsive to said signal processing circuit;
a vibration inducing member responsive to said oscillation device controller;
a status indicator responsive to said signal processing circuit; and
a tactile housing confining said vibration inducing member, said oscillation device controller, and said status indicator.

5. The device of claim 4, in which said at least one electrically responsive surface is a plurality of conductive pins.

6. The device of claim 2, in which said sensor assembly further comprising a communication port interacting with said signal processing circuit and communicating information between said signal processing circuit and a brainwave processing system.

7. The device of claim 3, in which said housing comprises a component chamber cooperating with a confinement cover, said component chamber supporting said sensor probe assembly, compressible electrically conductive member, and signal processing circuit and said confinement cover confining said sensor probe assembly, compressible electrically conductive member, and signal processing circuit within said component chamber.

8. The device of claim 1, in which said signal processing circuit comprising:
a printed circuit supporting a processor;
a differential amplifier interacting with said printed circuit member;
a reference signal communicating with said differential amplifier; and
a subject signal provided by said sensor probe assembly, when said sensor probe assembly is in electrical contact with a cranium of a subject, wherein said differential amplifier compares said reference signal to said subject signal and discards common signal patterns presented by said reference and subject signals to provide a native brainwave signal of the subject.

9. The device of claim 8, in which the signal processing circuit further comprising:
an analog to digital converter with a digital signal processing core responsive to said processor and interacting with said differential amplifier, said analog to digital converter processing said native brainwave signal provided by said differential amplifier and outputting a digital signal representative of said native brainwave signal;
an infinite impulse response filter interacting with said analog to digital converter to serve as a band pass filter for said digital signal; and
a memory communicating with said processor and storing a plurality of native brainwave signals, wherein said processor operates on a predetermined number of the plurality of said native brainwave signals to provide an equivalent root mean square value of the predetermined number of the plurality of said native brainwave signals, and further wherein the communication port communicating with the memory and responsive to the processor provides the equivalent root mean square value of the predetermined number of the plurality of said native brainwave signals to said brainwave processing system.

10. The device of claim 1, in which the brainwave processing system comprising:
a central processing unit communicating with the signal processing circuit;
a multi-channel input/output circuit electronically disposed between said central processing unit and said multi-channel input/output circuit;
a communication control circuit interacting with said central processing circuit and accommodating communication with remote devices; and
a memory means cooperating with said central processing unit to facilitate storage of an operating code, said operating code purposefully written to control operations of said signal processing circuit.

11. A method by steps comprising:
providing a plurality of sensor assemblies, in which each sensor assembly includes at least one electrically responsive surface;
supplying an oscillation device for communication with each said sensor assembly, wherein each said sensor assembly includes at least a signal processing circuit in electrical communication with the oscillation device;
agitating selectively at least one electrically responsive surface;
communicating performance measurement data from at least one of the plurality of sensor assemblies to a brainwave processing system; and
furnishing a ground reference, said ground reference interacting with the brainwave processing system, wherein a selected one of the plurality of sensor assemblies provides a reference signal for each of the remaining sensor assemblies, and in which each electrically responsive surface is in pressing contact with a cranium of a subject.

12. The method of claim 11, in which each said sensor assembly further comprising:
an electrically sympathetic member in electrical communication with said at least one electrically responsive surface;
an electrical element in electrical communication with said electrically sympathetic member; and
a signal conductor interacting with said electrical element and communicating signals facilitated by said at least one electrically responsive surface to said signal processing circuit.

13. The method of claim 12, in which said sensor assembly further comprising a housing confining said at least one electrically responsive surface, said electrically sympathetic member, said electrical element, said signal conductor and said signal processing circuit to collectively form said sensor assembly.

14. The method of claim 11, in which said oscillation device comprising:
an oscillation device controller responsive to said signal processing circuit;
a vibration inducing member responsive to said oscillation device controller;
a status indicator responsive to said signal processing circuit; and
a tactile housing confining said vibration inducing member, said oscillation device controller, and said status indicator.

15. The method of claim 14, in which said at least one electrically responsive surface is a plurality of conductive pins.

16. The method of claim 12, in which said sensor assembly further comprising a communication port interacting with said signal processing circuit and communicating information between said signal processing circuit and a brainwave processing system.

17. The method of claim 13, in which said housing comprises a component chamber cooperating with a confinement cover, said component chamber supporting said sensor probe assembly, compressible electrically conductive member, and signal processing circuit and said confinement cover confining said sensor probe assembly, compressible electrically conductive member, and signal processing circuit within said component chamber.

18. The method of claim 11, in which said signal processing circuit comprising:
a printed circuit supporting a processor;
a differential amplifier interacting with said printed circuit member;
a reference signal communicating with said differential amplifier; and
a subject signal provided by said sensor probe assembly, when said sensor probe assembly is in electrical contact with a cranium of a subject, wherein said differential amplifier compares said reference signal to said subject signal and discards common signal patterns presented by said reference and subject signals to provide a native brainwave signal of the subject.

19. The method of claim 18, in which the signal processing circuit further comprising:
an analog to digital converter with a digital signal processing core responsive to said processor and interacting with said differential amplifier, said analog to digital converter processing said native brainwave signal provided by said differential amplifier and outputting a digital signal representative of said native brainwave signal;
an infinite impulse response filter interacting with said analog to digital converter to serve as a band pass filter for said digital signal; and
a memory communicating with said processor and storing a plurality of native brainwave signals, wherein said processor operates on a predetermined number of the plurality of said native brainwave signals to provide an equivalent root mean square value of the predetermined number of the plurality of said native brainwave signals, and further wherein the communication port communicating with the memory and responsive to the processor provides the equivalent root mean square value of the predetermined number of the plurality of said native brainwave signals to said brainwave processing system.

20. The method of claim 11, in which the brainwave processing system comprising:
a central processing unit communicating with the signal processing circuit;
a multi-channel input/output circuit electronically disposed between said central processing unit and said multi-channel input/output circuit;
a communication control circuit interacting with said central processing circuit and accommodating communication with remote devices; and
a memory means cooperating with said central processing unit to facilitate storage of an operating code, said operating code purposefully written to control operations of said signal processing circuit.
